(19) United States
(12) Patent Application Publication
(10) Pub. No.: US 2006/0075514 A1
(43) Pub. Date: Apr. 6, 2006

(54) Transport across nuclear membranes by impulse transients

(76) Inventors: Thomas J. Flotte, Boston, MA (US); Apostolos G. Doukas, Belmont, MA (US)

Correspondence Address: FISH & RICHARDSON PC, P.O. BOX 1022, MINNEAPOLIS, MN, 55440-1022, US

(21) Appl. No.: 11/134,565

(22) Filed: May 20, 2005

Related U.S. Application Data
(60) Provisional application No. 60/573,165, filed on May 21, 2004.

(51) Int. Cl. C12N 15/01; A01N 1/02

(52) U.S. Cl. 800/21; 435/446; 435/284.1

(57) ABSTRACT

A method for temporarily permeabilizing a nuclear membrane to allow a molecule to enter a nucleus of a cell includes exposing the cell to a fluid medium containing the molecule; and causing, in the fluid medium an impulse having a peak pressure sufficient to permeabilize the nuclear membrane.

### RELATED APPLICATIONS

This application claims the benefit of priority of U.S. Provisional Patent Application Ser. No. 60/573,165, filed on May 21, 2004, the contents of which are incorporated herein by reference in their entirety.

### FIELD OF INVENTION

The invention relates to delivery of a compound into a cell, and in particular, to the delivery of a compound into the cell nucleus.

### BACKGROUND

In many cases, it is desirable to introduce molecules into the nucleus of a cell. For example, genetic material can carry out a useful function only if it is introduced into the nucleus of a cell.

Transport of small molecules (smaller than 17 kDa) across the nuclear membrane occurs by passive diffusion through the nuclear pore complexes. Larger molecules (larger than 41 kDa) require a nuclear localizing sequence and an active transport process to be transported into the nucleus. For exogenous compounds, such as dextrans, the nuclear envelope behaves like a molecular sieve with a functional pore radius of 5-6 nm. Dextrans molecules are spherical, hydrophilic, and inert molecules that have little tendency to be bound or degraded within cells. They are particularly suited for measuring translational mobility and transport between the cytoplasm and nucleus. Upon injection into the cytoplasm of the cell, it has been shown that dextrans molecules smaller than 17.5 kDa are distributed to the same concentration in the nucleus and the cytoplasm, whereas dextrans molecules larger than 41 kDa are found only in the cytoplasm.

At present, there are no known methods for directly permeabilizing the nuclear membrane. Known methods of introducing material into the nucleus are indirect. These methods generally involve permeabilizing the cell membrane to allow the material to enter the cytoplasm, and then relying on intra-cellular processes to transfer the material from the cytoplasm into the nucleus. One such method of introducing material into the nucleus is electroporation. In this method, a cell is placed in a high electric field. This field temporarily alters the permeability of the cell membrane so that material can be transported across the membrane and into the cytoplasm. When the field is removed, the permeability of the cell membrane is restored.

A difficulty with the foregoing method is that the high electric field can also destroy the cell. In addition, the electric field permeabilizes the cell membrane, but not the nuclear membrane. The delivery of the molecule the rest of the way into the nucleus thus relies on intra-cellular processes.,

### SUMMARY

The invention is based on the recognition that an impulse of pressure can be used to temporarily permeabilize a nuclear membrane.

In one aspect, the invention features a method for temporarily permeabilizing a nuclear membrane to allow a molecule to enter a nucleus of a cell. The method includes exposing the cell to fluid medium containing the molecule; and causing, in the fluid medium, an impulse having a peak pressure sufficient to permeabilize the nuclear membrane.

Embodiments of the invention include those in which causing the impulse includes generating a waveform having a peak pressure of at least 2 kilobar.

In some embodiments, causing an impulse includes providing a transducer for converting input energy into acoustic energy; placing the transducer in mechanical communication with the solution; and providing the transducer with input energy sufficient to generate the impulse wave form.

In these embodiments, the transducer can be selected to be a transducer that transforms input optical energy into acoustic energy. These embodiments include the optional step of illuminating the transducer with a laser pulse.

Some embodiments include placing the cell on the transducer.

Other embodiments include separating the transducer from the cell with a non-linear propagation medium. One such medium is a gel.

In those embodiments that include the use of a non-linear propagation medium, the properties of that medium can be selected to reduce the rise time of a pressure wave propagating through the medium.

Other embodiments include those in which the molecule is selected to include genetic material, and those in which the molecule is selected to include a therapeutic drug.

In another aspect, the invention includes a method of testing drugs by temporarily permeabilizing a nuclear membrane of a cell's nucleus using any of the foregoing methods.

In another aspect, the invention includes a system for introducing a molecule into a nucleus of a cell. Such a system includes a vessel for holding a fluid medium containing the molecule; a transducer in mechanical communication with the fluid medium for transducing input energy into an impulse transient in the fluid medium; and an energy source for providing the input energy.

In some embodiments, the energy source includes a laser configured to transmit a beam for ablating the transducer.

In other embodiments, the transducer includes a polystyrene plate having a first side in optical communication with the laser and a second side in mechanical communication with the fluid medium.

Certain other embodiments include those in which a non-linear propagation medium separates the fluid medium from the transducer. An example of a suitable non-linear propagation medium is a gel.

In those embodiments that incorporate a non-linear propagation medium, the properties of that medium can be selected to reduce the rise time of a pressure wave propagating therethrough.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

Other features and advantages of the invention will be apparent from the following detailed description, and from the claims.

### BRIEF DESCRIPTION OF THE DRAWINGS

FIG. 1 is a schematic of an apparatus for generating an impulse transient for permeabilizing a nuclear membrane.

FIG. 2 is a schematic of an apparatus similar to the one shown in FIG. 1, but with the addition of a gelatin layer.

### DETAILED DESCRIPTION

It has been found that an acoustic impulse having sufficiently high peak pressure and a short enough rise time temporarily permeabilizes both the cell membrane and the nuclear membrane. During this interval of permeability, molecules outside the cell membrane can cross into the cytoplasm, and molecules already in the cytoplasm can cross the nuclear membrane into the nucleus.

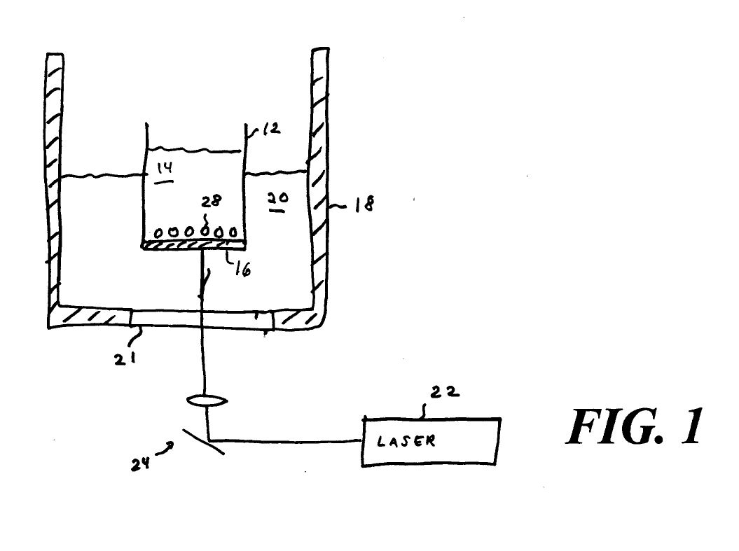

A system for transporting molecules into the nucleus, as shown in FIG. 1, includes an inner vessel 12 containing a solution 14 of molecules to be delivered into the nucleus. A transducer 16 is in mechanical communication with the interior of the inner vessel 12. As described herein, the transducer 16 is one that transforms optical energy into acoustic energy. However, the input energy source is not important, so long as the transducer 16 provides the necessary acoustic energy.

The inner vessel 12 is contained within an outer vessel 18 filled with water 20. The outer vessel 18 has a transparent portion 21 through which a beam produced by a laser 22 can be focused by an optical relay 24, e.g. a mirror and/or lens, onto the transducer 16.

In operation, a monolayer of cells 28 is placed adjacent to the transducer 16. The laser 22 then illuminates the transducer 16. The transducer 16 converts a portion of the laser energy into an impulse of pressure that propagates through the solution 14. The rise time and peak pressure of the impulse is selected to be sufficient to permeabilize the cell's nuclear membrane. A suitable peak pressure is on the order of 2 kilobar or greater.

The inner vessel 12 can be a 1 ml serological pipette having a 3 mm inner diameter. The transducer 16 can be a 1.5 mm thick black polystyerene plate attached to one opening of the pipette 12 by an epoxy adhesive. When ablated by a laser 22 on a first side thereof, the plate 16 carries a wave across to a second side opposite the first side. In this way, the polystyrene plate 16 functions as an optical-to-acoustic transducer 16.

The laser 22 can be a Q-switched ruby laser that radiates 28 nanosecond light pulses at a 694.3 nanometer wavelength. A suitable laser 22 is the RD-1200 laser manufactured by Spectrum Medical Technologies, of Natick, Mass. The optical relay 24 can include a spherical lens that focuses a 2 mm spot onto the transducer 16. This results in a spot having a mean energy density of 53 joules/cm².

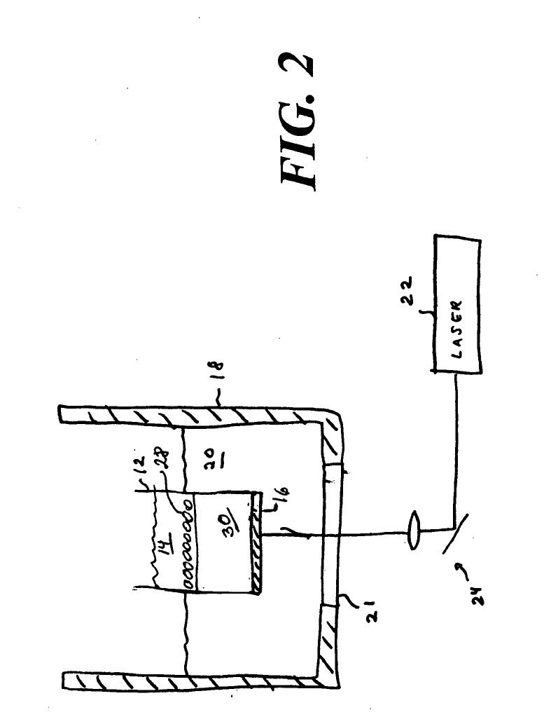

In another embodiment, shown in FIG. 2, a non-linear propagation medium, such as a gelatin layer 30, separates the cells 28 from the transducer 16. A gelatin layer 30 is useful because within it, high amplitude portions of an acoustic, or pressure wave propagate faster than low amplitude portions. This allows the pressure wave to develop a shorter rise time as it propagates across the gelatin layer 30.

The non-linear propagation of a pressure wave in a non-linear medium such as gelatin causes the leading edge of the waveform to sharpen. This results from the dependence of the wave's velocity on pressure. In particular, the wave's velocity increases along the leading edge of the pressure wave. This causes the rise time to decrease. On the other hand, linear attenuation, which increases as a function of frequency, attenuates predominantly the high frequency components, thereby causing the rise time to increase. The competing effects of the linear attenuation and the non-linear coefficient of the medium, the initial peak pressure, the initial rise time, and the distance traveled in the propagation medium will determine the final value of the rise time. The non-linear propagation in gelatin produces pressure transients having a rise time that is shorter than that generated by a pulsed laser alone.

The propagation distance L required for a plane wave to transform itself into a shock wave as it travels through the gelatin layer 30 can be estimated from non-linear acoustics by the relationship
L=lρc²/εP

where l is the spatial width of the pressure transient (i.e., its temporal duration multiplied by the sound velocity), ρ is the density of the gel, c is the sound velocity in the gel, ε is the non-linear coefficient, and P is the peak pressure. For the parameters of the desired pressure wave, and assuming that the non-linear coefficient of gelatin is the same as that of water (approximately 1.4), the propagation distance required (and hence the gel thickness) is approximately 3 mm under present experimental conditions.

### EXAMPLES

### Cell Preparation

Human peripheral blood mononuclear cells (“PBMC”) were used as target cells. The cells were prepared by first drawing blood in a heparinized syringe from healthy human volunteers. The blood was mixed with Dulbecco's phosphate buffered saline (PBS) without Ca²⁺ and Mg²⁺. The blood suspension was layered onto a ficoll-hypaque gradient in a 50-ml centrifuge tube. The tube was then spun at 1,200 RPM (200 g) for 40 minutes. The cells at the gradient/supernatant interface were collected and washed three times with PBS. The cell concentration was then adjusted to be 7×10⁶ cells/ml in PBS.

### Experimental Configurations

Individual wells were made of cut pieces of 1 ml plastic serological pipettes having a 3 mm inner diameter. Suitable pipettes were those manufactured by Becton Dickinson, N.J. The pipettes were sealed at one end with black polystyrene plates 1.5 mm in thickness. The plates were attached to the pipettes using epoxy adhesive.

Two configurations were used in the experiments. In FIG. 1, the cells 28 formed a monolayer on the bottom of the well 12 next to the polystyrene plate 16. In FIG. 2, the cells 28 were separated from the plate 16 by a solidified 3 mm gelatin column 30.

The gelatin column 30 was used to decrease the rise-time of the pressure transient by allowing the pressure waves to propagate through the gelatin 30. Previous experiments have shown that the rise time is an important parameter in the permeabilization of the cell membrane.

The gelatin column 30 in FIG. 2 was prepared as follows: A 5% gelatin solution prepared in PBS was injected into the wells by a 9 cm 22 G spinal needle syringe to a height of 3 mm. A suitable syringe is one manufactured by Becton Dickinson in N.J.

After the gelatin solidified at 4° C., the cells were injected into the wells 12 in both configurations, using another spindle needle syringe, and incubated at 4° C. for 30 minutes to form a monolayer at the top of the gelatin surface.

Then, 50 μl (micro-liters) of 124 μM (micro-molar) neutral fluorescein isothiocyanate (FITC)-dextran (FD-70, molecular weight 71,600 Da, (from Sigma, St. Louis, Mo.) in PBS was mixed in each well 12 with an equal volume (50 μl) of the cells to achieve a final concentration of 62 μM. Similarly, in the unirradiated controls, 50 μl of a solution of the cells in PBS was incubated with 50 μl of PBS (control 1) and 50 μl of FITC-dextran (control 2), respectively. The cells in the test sample were irradiated in the presence of the FITC-dextran.

### Exposure of Cells to Laser-Induced Pressure Transients

The cells were exposed to pressure transients generated by laser ablation of the polystyrene 16 as described above. A single 28 ns pulse from a Q-switched 694.3 nm ruby laser 22 (RD-1200, Spectrum Medical Technologies, Natick, Mass., USA) was steered via a series of mirrors and focused on the polystyrene target 16 by a spherical lens to a spot size 2 mm in diameter. The laser pulse was absorbed by the target to produce a single pressure transient. The cells 28 were not exposed to light. The fluence of the ruby laser 22 at the polystyrene plate 16 was 53 joules/cm². The peak pressure was estimated from previous studies using the same laser and the dependence of pressure on the laser fluence as reported in the literature. The peak pressure scales as the irradiance raised to the power of 0.7. Taking the ratio of 53 joules/cm² and 7 joules/cm², and raising to the power of 0.7 gives a factor of approximately 4. The peak pressure was thus approximately 2 kilobar. This peak pressure is the pressure generated in the target.

After irradiation, the cells 28 from tubes 12 of the same sample condition were pooled together. The gelatin layer 30 was thawed before aspiration by placing the cells in a 37° C. water bath for 2 minutes. All samples were washed three times with PBS and spun for 5 minutes each at 1200 RPM to remove extracellular FITC-dextran if any. After the third wash, the cells 28 were resuspended in 1 ml of PBS. The pooled samples were placed on ice. Approximately 4 hours elapsed from the time blood was drawn to the time when cells were ready for examination.

### Electroporation Experiments

For comparison, cells were subjected to electroporation. The electroporation source was an EasyjecT Optima (EquiBio, Kent, UK) that provided a 280 V/pulse, with a pulse duration of a few tens of milliseconds, an infinite shunt resistor, and a capacitor value of 1500 micorfarads. The 72 kDa FITC-dextran (as before) was added to the PBMC to achieve a final concentration of 62 μM. The cell suspension was vortexed and incubated at room temperature for 1 to 3 minutes. Then, 800 μL aliquots of cells were each placed into an electroporation cuvette (4 mm gap width, Eppendorf Scientific, Westbury, N.Y., USA). Within 30 seconds after electroporation, the exposed cell suspension was transferred to a centrifuge tube containing 10 ml of pre-warmed complete medium. The cells were spun at 1200 RPM for 10 minutes once and pellet resuspended in PBS.

### In Vitro Fluorescence Confocal Microscopy

Immediately before confocal microscopy, 1 μl of propidium iodide (PI) stock solution (1 mg/ml; Molecular Probes, Eugene, Oreg., USA) was added to a 50 μl aliquot of cell suspension for each sample. The suspension was then plated on a glass slide and covered by a cover slip. The samples were inspected 3 minutes after adding PI under a commercial confocal laser scanning microscope (Leica TCS-NT, Leica Lasertechnik GmbH, Heidelberg, Germany). Scans were taken with a 40-5 oil immersion objective (PL APO, 1.25-0.75, Leica, Germany) at different zoom levels. Percentages of cell loading and cell death with respect to the total cell population were then estimated from the resulting images.

### Data Analysis

An average fluorescence intensity per pixel was defined as the sum of fluorescence intensities in the designated area divided by the area, in pixels, after the background was subtracted. The background signal was derived from those viable cells that had not been loaded with the 72 kDa dextran in the same scans as the cells of interest. The procedure was carried out separately for the cytoplasm and the nucleus. The image processing was performed by standard software (IPLab Spectrum 2.4.01, Signal Analytics, Va., USA) on a MacIntosh IIvx computer (Apple Computers, Cupertino, Calif., USA). The average fluorescence intensity per pixel of the nucleus was compared to that of the cytoplasm using the paired t-test for cells treated by laser.

### Results

Propidium iodide (PI), a vital stain, was used to label dead cells by dye exclusion. Under a fluorescence confocal microscope, the non-viable cells appeared red and the viable cells loaded with FITC-dextran appeared green. In the first control group, which had been incubated with PBS, the viable cells showed intrinsic fluorescence only at a level considerably less than that of FITC fluorescence. The percentage of dead cells was approximately 15% of the total cell population. In the second control group, which had been incubated with the 72 kDa FITC-dextran, the dextran in the viable cells was localized in the cytoplasmic organelles rather than being found throughout the cytoplasm or in the nucleus. The percentage of dead cells in the second control group was similar to that in the first control group.

In the laser-irradiated test sample that had been incubated with the 72 kDa FITC-dextran, the percentage of cells that had taken up the dextran was 10%±5% when no gelatin was used and 25%±5% when the cells were placed on top of the 3-mm gel column. The dextran was nearly evenly distributed in both cytoplasm and nucleus of the cell. The percentage of dead cells rose to approximately 35% of the total cell population when the cells were exposed to a pressure transient. However, if only the dextran-loaded cells were considered, 99% of the cells remained viable.

In comparison, the fluorescence from the 72 kDa FITC-dextran was predominantly localized in the cytoplasm after electroporation, so that the loaded cells resembled “doughnuts.” The FITC-stained cells were usually found in clusters. Cellular debris was widespread.

The confocal microscopic impression was supported by quantification of the ratios of nuclear to cytoplasmic concentrations of dextran. The average fluorescence. intensity per pixel was proportional to the concentration of dextran molecules. Delivery with laser-induced pressure transients showed that the average fluorescence intensity per pixel in the nucleus (36±16) was slightly, but statistically significantly (p<0.05 by paired t-test) higher than that in the cytoplasm (29±13) with a ratio of nuclear to cytoplasmic concentrations of 1.2. The average background fluorescence intensity per pixel in the nucleus was 11±7, and that in the cytoplasm was 12±9.

The results clearly showed the presence of the 72 kDa dextran in the nucleus, following the pressure transients. This dextran would otherwise have been excluded from the nucleus upon cytoplasmic introduction, as is the case in electroporation. It is important to note that 99% of the cells that showed cytoplasmic and nuclear loading remained viable.

The present experiments indicate that permeabilization of the nuclear envelope requires a higher pressure gradient (higher peak pressure, shorter rise time or both) than permeabilization of the plasma (or cell) membrane. The fact that higher cell killing was observed at approximately 35% is consistent with this conclusion. It should be pointed out, however, that even this level of cell killing (35%) is less than the level of cell killing observed during electroporation.

### Applications

In gene therapy, it is hoped that human disease might be treated by transfer of genetic material into specific cells of a patient. Pressure transients as described herein provide a potentially powerful tool for gene delivery. Photophonoporation of nuclear envelopes offers unique characteristics compared to other nonviral DNA transfection methods, such as electroporation, ligand-DNA conjugates, adenovirus-ligand-DNA conjugates, lipofection, direct injection of DNA, and calcium phosphate precipitation. The advantages may include in vivo or in vitro application, spatial and temporal localization, either local or distant exposure of transients, and high levels of cell survival.

The methods described herein may also provide an opportunity for new classes of drugs. For example, one constraint in drug design is that the drug molecules be small enough to cross the cell membrane. It should be possible to use this approach in combination with fiberoptic shock wave generators and catheter technology for novel drug and gene therapy in the cardiovascular system. Potentially, this technology can deliver anti-sense oligonucleotides to interrupt signals, such as the signal for smooth muscle proliferation following balloon angioplasty. This approach may also have applications in cell biology for introduction of molecules into large numbers of cells while maintaining a high level of cell survival.

### Other Embodiments

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

1. A method for temporarily permeabilizing a nuclear membrane to allow a molecule to enter a nucleus of a cell, the method comprising:
exposing the cell to a fluid medium containing the molecule, the cell having a nucleus surrounded by a nuclear membrane; and
causing, in the fluid medium, an impulse having a peak pressure sufficient to permeabilize the nuclear membrane.

2. The method of claim 1, wherein causing an impulse comprises generating a waveform having a peak pressure of at least 2 kilobar.

3. The method of claim 1, wherein causing an impulse comprises:
providing a transducer for converting input energy into acoustic energy;
placing the transducer in mechanical communication with the fluid medium; and
providing the transducer with input energy sufficient to generate the impulse wave form.

4. The method of claim 3, further comprising selecting the transducer to be a transducer that transforms input optical energy into acoustic energy.

5. The method of claim 4, further comprising illuminating the transducer with a laser pulse.

6. The method of claim 3, further comprising placing the cell on the transducer.

7. The method of claim 3, further comprising separating the transducer from the cell with a non-linear propagation medium.

8. The method of claim 7, further comprising selecting the non-linear propagation medium to be a gel.

9. The method of claim 7, wherein the properties of the non-linear prpagation medium are selected to reduce the rise time of a pressure wave propagating from the transducer.

10. The method of claim 1, further comprising selecting the molecule to include genetic material.

11. The method of claim 1, further comprising selecting the molecule to include a therapeutic drug.

12. A method of testing drugs, the method comprising permeabilizing a nuclear membrane as recited in claim 1.

13. A system for introducing a molecule into a nucleus of a cell, the system comprising:
a vessel for holding a fluid medium containing the molecule;
a transducer in mechanical communication with the fluid medium for transducing input energy into an impulse transient in the fluid medium; and
an energy source for providing the input energy.

14. The system of claim 13, wherein the energy source comprises a laser configured to transmit a beam for ablating the transducer.

15. The system of claim 14, wherein the transducer comprises a polystyrene plate having a first side in optical communication with the laser and a second side in mechanical communication with the fluid medium.

16. The system of claim 13, further comprising a non-linear propagation medium separating the fluid medium from the transducer.

17. The system of claim 16, wherein the properties of the non-linear propagation medium are selected to reduce the rise time of a pressure wave propagating therethrough.

18. The system of claim 16, wherein the non-linear propagation medium comprises a gel.
